(19) United States
(12) Patent Application Publication
(10) Pub. No.: US 2021/0316027 A1
(43) Pub. Date: Oct. 14, 2021

(54) SECURE CONTAINER AND METHOD OF OPERATION SAME

(71) Applicant: LokPort Corp., Rock Springs, WY (US)

(72) Inventor: Sean O'Keefe, Rock Springs, WY (US)

(73) Assignee: LokPort Corp., Rock Springs, WY (US)

(21) Appl. No.: 17/229,728

(22) Filed: Apr. 13, 2021

Related U.S. Application Data
(60) Provisional application No. 63/009,394, filed on Apr. 13, 2020.

(51) Int. Cl. A61L 2/24; A47G 29/30; A61L 2/22; A61L 2/10

(52) U.S. Cl. CPC A61L 2/24; A47G 29/30; A61L 2202/122; A61L 2/10; A61L 2202/14; A61L 2/22

(57) ABSTRACT

An apparatus and method for securely and safely storing an item includes a container having a plurality of walls configured to define an interior space of the container and an entry panel positioned in at least a portion of one of the plurality of walls and operable to open and reveal the interior space of the container. An open/close controller is coupled to the entry panel and configured to control the opening and closing of the entry panel. A germicidal controller is positioned within the interior space of the container and configured to provide a germicide to the interior space. The container also includes circuitry coupled to the open/close controller and germicidal controller and a memory coupled to the circuitry having a plurality of instructions executed by the circuitry. In response to receiving a delivery signal for an item to be inserted into the container, the delivery signal including at least a first unique identifier, the open/close controller is controlled to open the entry panel if the first unique identifier included in the delivery signal is verified and then close the entry panel after the item has been placed in the interior space of the container. The germicidal controller is controlled to provide the germicide to the interior space of the first compartment entry panel of the container has been closed. In response to receiving a pickup signal for an item to be removed from the container, the pickup signal including at least a second unique identifier, the open/close controller is controlled to open the entry panel if the second unique identifier included in the pickup signal is verified. The open/close controller is controlled to close the entry panel after the item has been removed from the interior space of the container.

### BACKGROUND

### Field

This disclosure relates to field of containers or storage units and to an apparatus and method for securely and safely receiving, storing, and providing items.

### Description of the Related Art

Containers or storage spaces for receiving mail, packages, or other items typically have few if any security features to protect against unauthorized persons accessing and taking the items for themselves. The standard mailbox may have no more than a panel or door that can be freely opened or closed by anyone, not just the delivery person and the owner of the mailbox, and in fact may have a permanent opening that does not even shield its contents from view. Moreover, with the proliferation of Amazon and other delivery services providing everything from groceries to restaurant deliveries to clothing to electronics, the delivered items are often left unattended on a front porch, driveway, or stoop with no protection at all from passers by taking the items, sometimes referred to as porch pirates. It would therefore be desirable to have a container, storage, or other receptacle that includes security features to minimize or eliminate risks from thievery.

At the same time, the Covid 19 pandemic has raised concerns about contact with materials or surfaces that may be supporting viruses, bacteria, or other germs. Science has shown that some bacteria and infectious diseases can last for days and perhaps longer on certain surfaces including paper, cardboard, plastic, packaging labels, Styrofoam, stamps, and tape. These concerns have made it desirable that mail, packages, and other items delivered to containers, storage, or other receptacles minimize the risk of transmission from contact with surfaces therein, both for those delivering the item to the container or storage and for those picking up the item from it.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 shows an exemplary container according to an embodiment;

FIG. 2 shows an exemplary container with an open entry panel according to an embodiment;

FIG. 3 is a flow chart for operating a container according to an embodiment in which an item is placed in the container; and

FIG. 4 is a flow chart for operating a container according to an embodiment in which an item is removed from the container.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, the subject technology is not limited to the specific details set forth herein and may be practiced using one or more implementations. In one or more instances, structures and components are shown in simplified form in order to avoid obscuring the concepts of the subject technology.

In the drawings referenced herein, like reference numerals designate identical or corresponding parts throughout the several views or embodiments.

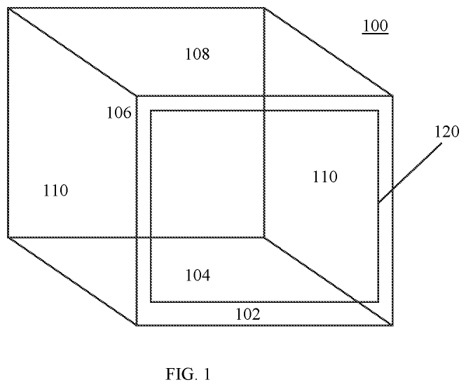

FIG. 1 shows an exemplary container for secure, safe, and contactless transfer of items from a supplier to a customer according to an embodiment. As shown in FIG. 1, a container 100 includes walls 102, 104, 106, 108, 110, and an entry panel 120. The container 100 can have a variety of shapes and sizes and can be made from a variety of materials. For example, the container 100 can be shaped like a cube, a rectangular box, or any other shape that is capable of holding and storing items. The size of the container 100 is similarly flexible and can have, for example, a 1 foot, 2 foot, 3 foot, 1 meter, or any other length for a side of the container 100 as desired to hold and store items. The materials for the container 100 can be any suitable material, such as aluminum, steel, other metals, alloys, fiberglass, or any other material that makes it difficult for unauthorized users to access the interior space of the container 100. Although referred to as container 100, other terms may also be used such as box, storage, receptacle, locker, vessel, holder, repository, canister, drum, or case, for example.

Wall 102 is a front wall of container 100, wall 104 is a bottom wall of container 100, wall 106 is a back wall of container 100, wall 108 is a top wall of container 100, and walls 110 are sidewalls of container 100. Entry panel 120 can be formed in or out of a portion of, for example, front wall 102, although it can also be formed in or out of a portion of the other walls of container 100. Alternatively, entry panel 120 can also form an entire wall of container 100. As explained in more detail herein, entry panel is configured and designed to open to allow access to an interior space of container 100, preferably only when receiving a signal authorizing its opening. The materials for container 100 described above can also be used for each of walls 102, 104, 106, 108, 110 and entry panel 120 or some combination of those materials. For example, entry panel 120 may use a different material than the rest of the container 100. Moreover, although shown with only one entry panel, container 100 may include more than one entry panel. Container 100 can also be configured to have more than one interior space where each interior space can have its own separate entry panel, but each interior space is inaccessible to any other interior space. In that configuration, container 100 can have multiple storage spaces with separate and independent access through corresponding entry panels.

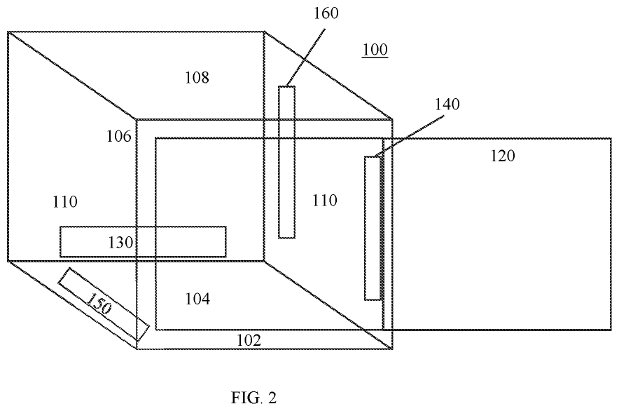

FIG. 2 shows an exemplary container with an open entry panel according to an embodiment. As shown in FIG. 2, when entry panel 120 of container 100 is open, it reveals an interior space of container 100 and the space in which one or more items can be placed, stored, and removed. Within the interior space, container 100 includes a germicidal controller 130, an open/close controller 140, a temperature controller 150, and container controller 160. Each of these components in the interior space of container 100 can be attached to an interior wall by any of a variety of way including adhesive, screws, nuts and bolts, welds, or any other way sufficient to attach them to an interior wall of the container 100. Although shown as separate components, the germicidal controller 130, open/close controller 140, temperature controller 150, and container controller 160 can be combined into a single component or some sub-combination thereof.

The germicidal controller 130 is configured to reduce and/or eliminate the risk of germs being present in the container 100. The germicidal controller 130 can include, for example, an ultraviolet (UV) light that acts to provide germicidal irradiation of all surfaces and contents of the container 100. UV germicidal irradiation (UVGI) is an example of a disinfection method that uses short wavelength ultraviolet (UV-C) light to kill or inactivate microorganisms by destroying nucleic acids and disrupting their DNA, leaving them unable to perform vital cellular functions. To minimize risks associated with the use of UV-C light, the germicidal controller can preferably use “Far UVCI Light” emitters that are known to be safer for and not harmful to the environment. Such emitters include, for example, UVCI LED's. In addition, to ensure the UVGI is able to irradiate all surfaces, the germicidal controller 130 can include multiple sources of UVGI positioned at multiple locations within container 100. The germicidal controller 130 can also use other forms of germicide instead of or in addition to UVGI. For example, the germicidal controller 130 can include a germicidal spray or gas that has a germicidal function such as ones including alcohol, hydrogen peroxide, chlorine, formaldehyde, glutaraldehyde, and/or phenol.

The germicidal controller 130 can also include circuitry, such as a microprocessor, microcontroller, CPU, memory, RAM, and/or ROM, that can be configured to receive control signals and, in response to those control signals, control the UVGI, spraying of the germicide, or other form of germicide to sanitize the container 100 in accordance with the received control signals. Through activation of the germicidal controller 130, it is possible to sanitize container 100 including its interior space, the interior walls, and any items stored therein. Doing so creates a safe transfer of the items and further assists in the safety of the supply chain and logistics protocols by mitigating or eliminating exposure to disease-causing germs during the transfer of the item from the supplier to a customer or end users.

The opening of the entry panel 120 can be controlled by the open/close controller 140, which can be configured to move the entry panel 120 between an open position and a closed, locked position. The open/close controller 140 can include a motorized mechanism having, for example, a gas or hydraulic rod, a spring, a solenoid, and/or a servo unit. In addition, the open/close controller can include a lock, such as a ratchet lock or deadbolt lock, that further secures the entry panel when in a closed position. The open/close controller 140 preferably includes electronic circuitry, such as a microprocessor, microcontroller, CPU, memory, RAM, and or ROM, configured to control the opening and closing of the entry panel 120 in response to a received signal, as will be explained in more detail herein. The container 100 can also include other electronic components including, for example, one or more cameras, a keyboard, an entry pad, biometric reader, microphone, speaker, RFID, and/or LED lights or other form of indicators. These other electronic components can assist in controlling access to the container 100 and the opening and closing of the entry panel 120, as will be explained in more detail herein.

The container 100 can also include the temperature controller 150. The temperature controller 150 preferably includes both heating and cooling elements that can control the temperature within the container 100 to be set to a particular or desired temperature setting. The temperature setting can be adjusted depending on the type of item provided within the container 100. For example, for food items that have just been prepared, the temperature controller 150 can be configured to be set to a higher than ambient temperature to keep the food items warm. Alternatively, if the food items are fresh produce or meats, then the temperature controller 150 can be configured to be set to a lower than ambient temperature to keep the food items cool. The temperature controller 150 can also include circuitry, such as a microprocessor, microcontroller, CPU, memory, RAM, and or ROM, that can be configured to receive a control signal and, in response to those control signals, control the heating and/or cooling elements to set the temperature within the interior space of the container 100 to a setting in accordance with the received control signal.

The container 100 can additionally include the container controller 160, which preferably includes circuitry, such as a microprocessor, microcontroller, CPU, memory, RAM, and/or ROM, that can be configured to control all of the operations of the container 100 including the operation of the germicidal controller 130, the open/close controller 140, and the temperature controller 150. In addition to this operational functionality, container controller 160 preferably includes transmitters and receivers that can transmit and receive data and signals over a variety of transmission paths including cellular, Wi-Fi, Bluetooth, NFC via IoT or any other form of wired or wireless transmission. Container controller 160 can also include one or more cameras, a keyboard, biometric reader, microphone, speaker, RFID, and/or GPS circuitry.

The container 100 can be located at a house, apartment building, office building, mall, shopping center, or any location in which an item can be delivered, stored, and picked up. With its design and configuration, the container 100 can securely and safely enable customers or end users to order or have items delivered to the container, opened only when the container 100 receives a proper authorizing signal (both to put in an item and to take out the item), and keep surfaces and items within the container 100 clean and sanitized to reduce or eliminate the risk of transmission of germs between the supplier of the item and the receiver of it. These features can be met by configuring the germicidal controller 130, open/close controller 140, temperature controller 150, and container controller 160 as explained herein.

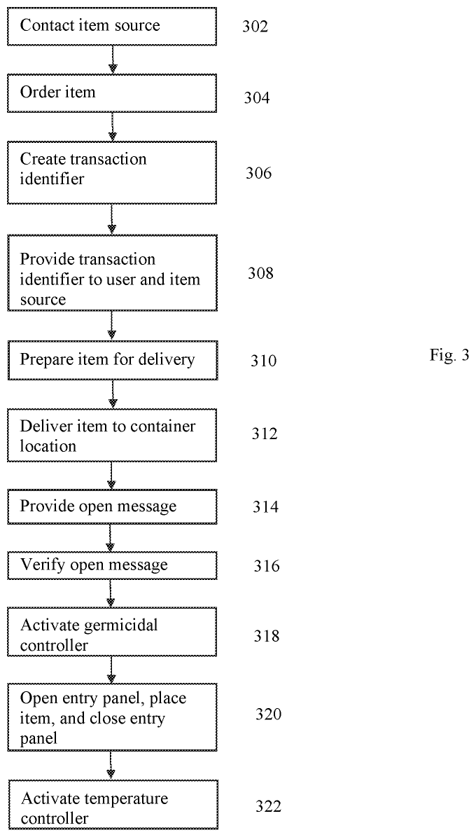

FIG. 3 is a flow chart for operating a container according to an embodiment in which an item is placed in the container. The process of FIG. 3 can be used in conjunction with any business that provides items that can be stored and delivered to a container and picked up by the customer or end user ordering the item. As shown in FIG. 3, a user, such as a customer or other end user, contacts a business to make an order and arrange delivery of an item (step 302). The user can contact the business through, for example, a website using a mobile device, cell phone, desktop computer, a laptop, tablet, or other computing device, or through an application running on such a device. Alternatively, the user may contact the business by phone or email. If it is a returning or existing user, the business may automatically recognize the user through cookies on the user's device and/or through a login or registration process completed by the user. The information associated with the user through that recognition can include the user's name, address, contact number, payment information, past orders, and preferences. These preferences can include identifying information about the user including, for example, biometric information like a facial ID or fingerprint, a code created by the user, or other data that uniquely identifies the user. These preferences can also include information about how the user prefers to have items delivered, a preferred delivery address, and a preferred identifier to use for accessing the container 100 holding a user's ordered items (e.g., using facial ID or a transaction ID).

The user contacting the business can make an order for one or more items from the business (step 304). The items can be, for example, packages, mail, food, or any other type of consumer item that may fit within container 100. The order may be submitted through the business's website, application, or orally through a phone call. The order preferably includes information identifying the requested items, the location to which to deliver the items, customer information, and any other information as may be needed to complete the order and make the delivery. The business receives and stores the order information for reference in fulfilling the order.

In response to the order, unique identifying information is created as a transaction identifier for the order made by the user (step 306). The unique identifying information created for the transaction identifier can be, for example, a random number, a value created based on identification information of the user, a QR code, or any other unique value that can be used to uniquely identify the transaction corresponding to the order made by the user. In addition, different unique identifying information can be created and assigned to the business and the user.

The transaction identifier is provided to the user and the business (step 308). The transaction identifier provided can be the same to both the user and the business or different ones but relating to the same order. If the order was made through the business's website or application, the transaction identifier can be provided directly to the user's and business' computing devices through the corresponding website or application, sent by text, or sent by email. If the order has been made by phone, then an operator at the business can provide the user with the transaction identifier by phone.

Once the order has been completed and the transaction identifier has been provided to the user and the business, the business prepares the requested item(s) for delivery (step 310). This preparation may include, for example, collecting items from storage or shelves or preparing or collecting food items, and then placing the requested items into some form of packaging.

When the business has completed preparing the requested items, the item is picked up and delivered to the location of the container 100 (step 312). When the person delivering the ordered item arrives at the location of the container 100, the entry panel 120 of the container 100 is closed. To get the entry panel 120 to open, the delivery person can send a message or provide authorizing information to the mobile transport (step 314). The message sent by the employee can include information about the delivery including, for example, the transaction identifier or other unique identifier that has been provided to the business when the order was made. In this manner, the transaction identifier or other unique identifier can operate like a one-time use code that enables the delivery person for the business to gain access to the container 100 for that transaction only.

The delivery person can send the message from a computing device, such as a mobile phone or tablet, and the message is received by a receiver of the container controller 160. Alternatively, instead of sending a message from a computing device, the delivery person can enter a code on a keypad located on the container 100 or have biometric information examined such as by a facial scan, a retinal scan, or a fingerprint scan to ensure that the delivery person is authorized to place items into the container 100.

Based on the received message or authorization, the container 100 verifies the received message includes the proper information to enable the entry panel 120 to be opened (step 316). To make this verification, the container 100 can receive the order information and the transaction identifier or other unique code or identifying information (such as biometric) at the time the user makes the order and use that information to make the verification in response to the received message. For example, the user may have an application operating on a mobile phone, tablet, laptop, or desktop computer that, in response to the completion of the order, sends the relevant information to the container 100, such as via WiFi or a cellular connection. The container controller 160 can be configured to perform the verification.

If verified, then prior to opening the entry panel 120, the germicidal controller 130 can be activated to sanitize the container 100 (step 318). For example, if the container controller 160 confirms the verification, the container controller 160 can send a signal to the germination controller 130 to be activated, and the germicidal controller 130 activates the germicidal action in response to the signal, such as by activating the UVGI source to irradiate all the surfaces of the interior the container 100. By activating the germicidal controller 130 at this time, the container 100 will already have been sanitized before the delivery person is exposed to it and thereby minimize or eliminate the risk of the delivery person being exposed to germs.

Once the germicidal action is complete, the entry panel 120 is opened, the delivery person places the item(s) in the container 100, and the entry panel 120 is closed (step 320). For example, the container controller 160, having verified the received message, sends a signal to the open/close controller 140 to open the entry panel 120 of the container 100. In response to the signal, the open/close controller 140 opens the entry panel 120 of the container 100. Alternatively, the germicidal controller 130 can be configured to send a signal to the open/close controller 140 to open the entry panel 120 of the container 100 once the germicidal action is complete. Once placed into the container 100, the entry panel 120 is closed. The signal for the open/close controller 140 to close the entry panel 120 can be provided by the container controller 160. The container controller 160 can send the signal to the open/close controller 140 in response to a message or authorization from the delivery person, based on the elapsing of a specific time period, or based on a detection system that determines the items have been placed inside the compartment. The detection system can include, for example, motion detectors that send signals to the container controller 160, and the container controller 160 can be configured to interpret the signals from the motion detectors to recognize when the item has been placed inside the container 100 and correspondingly that the entry panel 120 can be closed.

In addition to opening and closing the entry panel 120 for insertion of the ordered item, temperature control can be activated (step 322). For example, based on the type of items placed in the container 100, the container controller 160 can send a signal to the temperature controller 150 to control the temperature inside the interior space of the container 100 to be set to an appropriate setting. In response to the signal, the temperature controller 150 can set the interior temperature to keep the items warm or to keep the items cold. On the other hand, for non-perishable items and items having no need for maintaining a temperature warmer or cooler than the ambient temperature, the container controller 100 need not send any signal to the temperature controller 150. It is also possible that when the need for a temperature controller is known in advance, the container controller 160 can send a control signal to the temperature controller 150 to start warming or cooling for a period of time before items are placed in the container 100. The container controller 160 can also be configured to determine the type of item present in the container 100 and send a signal to the temperature controller 150 based on the determination. The determination can be made, for example, based on information in the order or based on image recognition processing implemented in the container controller 160.

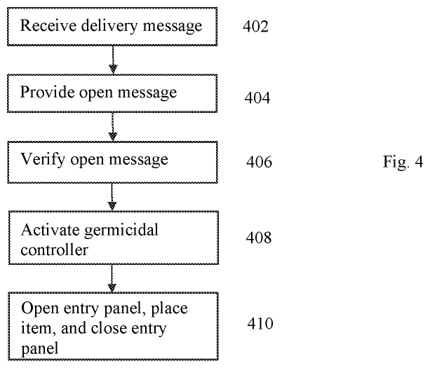

FIG. 4 is a flow chart for operating a container according to an embodiment in which an item is removed from the container. Like the process of FIG. 3, the process of FIG. 4 can be used in conjunction with any business that provides items that can be stored and delivered to a container and picked up by the customer or end user ordering the item. As shown in FIG. 4, the user can receive a message that the item is present in the container 100 (step 402). For example, the container controller 160 can send a message to the user that includes information about the delivery of the item, the availability to pick up the item, and optionally resend the transaction identifier or other unique identifier for the user to use to access the container 100. The message can be sent from a transmitter of the container controller 160 to any of a variety of computing devices of the user including PC's, laptops, and mobile devices such as mobile phones or tablets. The messages can be text messages, emails, or voice messages.

To access the item when the user arrives at the container 100, the user can send a message or provide authorizing information to the container 100 (step 406). The message sent by the user can include information about the delivery including, for example, the transaction identifier or other unique identifier that enables the container controller 160 to determine whether to open the entry panel 120. The message can be sent from a computing device, such as a mobile phone or tablet, and received by a receiver of the container controller 160. Alternatively, instead of sending a message from a computing device, the user can enter a code on a keypad located on the container 100, have a QR code scanned, or have biometric information examined such as by a facial scan, a retinal scan, or a fingerprint scan to ensure that the user is authorized to access the container holding the ordered items.

Based on the received message or authorization, the container 100 verifies the received message includes the proper information to enable the entry panel 120 to be opened (step 408). To make this verification, the container 100 can receive the transaction identifier or other unique code or identifying information (such as biometric) at the time the user had made the order and use that information to make the verification in response to the received message. For example, the user may have an application operating on a mobile phone, tablet, laptop, or desktop computer that, in response to the completion of the order, sends the relevant information to the container 100, such as via WiFi or a cellular connection. The container controller 160 can be configured to perform the verification.

If verified, then prior to opening the entry panel 120, the germicidal controller 130 can be activated to sanitize the container 100 (step 410). For example, if the container controller 160 confirms the verification, the container controller 160 can send a signal to the germination controller 130 to be activated, and the germicidal controller 130 activates the germicidal action in response to the signal, such as by activating the UVGI source to irradiate all the surfaces of the interior the container 100. Activating the germicidal controller 130 at this time provides similar benefits including ensuring that the container 100 and the delivered item will already have been sanitized before the user is exposed to it.

Once the germicidal action is complete, the entry panel 120 is opened, the user removes the item(s) from the container 100, and the entry panel 120 is closed (step 412). For example, the container controller 160, having verified the received message, sends a signal to the open/close controller 140 to open the entry panel 120 of the container 100. In response to the signal, the open/close controller 140 opens the entry panel 120 of the container 100. Alternatively, the germicidal controller 130 can be configured to send a signal to the open/close controller 140 to open the entry panel 120 of the container 100 once the germicidal action is complete. Once placed into the container 100, the entry panel 120 is closed. The signal for the open/close controller 140 to close the entry panel 120 can be provided by the container controller 160. The container controller 160 can send the signal to the open/close controller 140 in response to a message or authorization from the user, based on the elapsing of a specific time period, or based on a detection system that determines the items have been removed from inside the compartment. The detection system can include, for example, motion detectors that send signals to the container controller 160, and the container controller 160 can be configured to interpret the signals from the motion detectors to recognize when the item has been removed from inside the container 100 and correspondingly that the entry panel 120 can be closed.

The activation of the germicidal controller 130 to provide a germicide, such as UV light, to the interior space of the container can be applied at times other than those described in the processes of FIGS. 3 and 4. For example, the germicidal controller 130 can be configured on its own or in response to signals from the container controller 160 to activate after a certain period of time, such as every three hours. Alternatively, the activation of the germicidal controller 130 can be every time the entry panel of the container 100 is closed.

The order, delivery, and pickup process of FIGS. 3 and 4 provide multiple benefits that make the order, delivery, and pickup convenient, safe, and cost-effective for businesses and users ordering items. These benefits include, for example, the ability to apply germicides to the container 100 that are used to store and deliver items with the use of germicidal controller 130 before the container 100 is accessed by delivery persons or users receiving a delivered item. This ability minimizes or eliminates the risk of transmission of viruses and bacteria for both the delivery person and the users receiving the delivered item. Moreover, with only one person placing the items in the container 100 and only one person later removing the items, people-to-people contacts are avoided. By providing temperature controller 150, the container 100 can store items, particularly foodstuffs, while maintaining an appropriate temperature for those items, both above and below the ambient temperature.

The order, delivery, and pickup process of FIGS. 3 and 4 can provide enhanced security for ordered items. Items delivered by mail or delivery services are typically placed in an unsecure manner, such as in a mailbox, at a front door, or on a driveway, or require the recipient to be present at the time of delivery. With the process using the container 100, the container 100 remains closed and locked except when the container receives the proper message to open the entry panel 120. Limiting access to the container 100 in this manner ensures that unauthorized persons are unable to open the container 100 and remove the delivered item, thus minimizing or eliminating the risk of thievery from so-called porch pirates. Since delivered items may also include valuable identifying information about the recipient beyond name and address, such as account information, shielding the delivered item from unauthorized persons provides more security for the user.

The message received by the container 100 for opening the entry panel 120 goes through a verification performed, for example, by the container controller 160 and only signals the open/close controller 140 to open the entry panel 120 if the received message is verified. In addition, the received message verified by the container controller 160 can be a unique, one-time code or identifier that is provided exclusively to the business providing the item and the user receiving the item. The unique code or identifier ensures only the business providing the item and the user ordering the item are capable of opening the container 100. Moreover, by using a unique, one-time code or identifier, the delivery person will only be able to access the container 100 for that particular delivery. Similarly, if the container 100 is shared by multiple users, the user making the order will only be able to access the container 100 for that particular order.

In the example of FIGS. 1 and 2, the various controllers are built into the container 100. It is possible instead to provide a kit to convert a container into a container having some or all of the functionality of the container 100 as described above. The container to be converted by the kit preferably includes a complete enclosure for storing an item and an entry panel that can be controllably opened and closed. The kit can include, for example, a germicidal controller, open/close controller, temperature controller, and container controller having some or all of the same functionality and components as described above. The kit can be a simplified version that combines two or more controllers into one controller and/or combines the functionality of two or more controllers into one controller. The kit can also include instructions for installing the controllers in the container including the manner for attaching each controller to the container and attaching the mechanical structure for controlling the opening and closing of the entry panel and the locking of the entry panel when closed.

The security for the container 100 can be further enhanced by providing an alarm system. The functionality for the alarm system can be built into one of the controllers, such as the container controller 160, or can be a separate and independent controller that is coupled to the container controller 160. The alarm system can be configured to provide an alarm or other type of alert in response to an attempt to access the container 100 without authorization. For example, an unauthorized user may attempt to access the container by providing a false message to the container, such as by using a mobile phone, tablet, laptop, desktop, or other type of computing and/or electronic device. In response to the false message, the container controller 160 can be configured to detect the false message, perform a verification, and confirm the false message does not provide authorization. The container controller 160 can also keep a count of the number of messages received that cannot be verified and issue an alarm when the number exceeds a threshold. The threshold can be set to one or more messages as desired. Alternatively, or in addition to this detection of unauthorized access, the container 100 can include motion and/or movement sensors that detect movement in and around the container 100 as well as movement of the container 100, such as may occur when an unauthorized person attempts to physically open the container. The container controller 160 can be configured to receive the signals from these motion/movement sensors, determine whether an unauthorized access is being attempted, and, if so, issues an alarm.

The issued alarm can be an audible and/or visual alarm provided at the container 100. The issued alarm can also include or only include sending an alarm message to the user of the container 100. The alarm message can be sent, for example, as a text, an email, a voice call, or other form of electronic messaging to the user. The alarm message can also be sent to an enforcement agency, such as a police department or a security company.

The alarm system can include a camera that is activated to capture activity around and in the vicinity of the container 100. The camera can be activated when motion sensors around the container 100 detect movement near the container 100. The camera can alternatively be activated when movement sensors detect movement of the container 100. Recordings from the camera can be provided, for example, to the user or to an enforcement agency to assist in apprehending persons involved in attempting unauthorized access to the container 100.

The container 100 can also be configured to be mobile. A set of wheels can be attached to the bottom wall 104 of the container 100 to make it mobile. Additionally, a motor and a transmission or other driving mechanism can also be included with the container 100 and coupled to the wheels to enable the container to be moved from location to location automatically without human interaction to impel the movement. The container controller 160 can include a GPS circuit and be configured to move to a location based on a received signal. For example, within an office space where sensitive documents are shared, a first user could securely and safely place the sensitive document in the container 100, provide a signal indicating the location within the office to which to bring the sensitive document, move to that location, and then only open the container when the intended recipient provides the proper authorizing message.

As noted previously, the container 100 can include multiple independent interior spaces each having separate and independently controller entry panels defining individual compartments within container 100. The individual compartments can be sized differently and have different functionality and capabilities. For example, some compartments may have a temperature controller while others do not, thereby enabling food items to be stored in temperature-controlled compartments while other items not needing temperature control can be stored in compartments without temperature controllers.

Various embodiments of the invention are contemplated in addition to those disclosed hereinabove. The above-described embodiments should be considered as examples of the present invention, rather than as limiting the scope of the invention. In addition to the foregoing embodiments of the invention, review of the detailed description and accompanying drawings will show that there are other embodiments of the present invention. Accordingly, many combinations, permutations, variations and modifications of the foregoing embodiments of the present invention not set forth explicitly herein will nevertheless fall within the scope of the present invention.

1. A container for secure storage of an item, comprising:
a plurality of walls configured to define an interior space of the container;
an entry panel positioned in at least a portion of one of the plurality of walls and operable to open and reveal the interior space of the container;
an open/close controller coupled to the entry panel and configured to control the opening and closing of the entry panel;
a germicidal controller positioned within the interior space of the container and configured to provide a germicide to the interior space;
circuitry coupled to the open/close controller and germicidal controller; and
a memory coupled to the circuitry comprising a plurality of instructions that, when executed by the circuitry, cause the container to:
receive a delivery signal for an item to be inserted into the container, the delivery signal including at least a first unique identifier;
control the open/close controller to open the entry panel if the first unique identifier included in the delivery signal is verified;
control the open/close controller to close the entry panel after the item has been placed in the interior space of the container;
control the germicidal controller to provide the germicide to the interior space of the first compartment after closing the entry panel of the container;
receive a pickup signal for an item to be removed from the container, the pickup signal including at least a second unique identifier;
control the open/close controller to open the entry panel if the second unique identifier included in the pickup signal is verified; and
control the open/close controller to close the entry panel after the item has been removed from the interior space of the container.

2. The container of claim 1, further comprising a temperature controller configured to individually control a temperature setting within the container, the temperature setting being below, at, or above an ambient temperature,
wherein the memory further comprises instructions executed by the circuitry to cause the container to:
determine a type of the item placed in the interior space of the container; and
control the temperature controller to set the temperature setting for the container according to the determined type of the item.

3. The container of claim 1, wherein the germicidal controller includes one or more UV light sources, the memory further comprising an instruction executed by the circuitry to cause the container to control the germicidal controller to activate the one or more UV light sources after the entry panel of the container is closed.

4. The container of claim 1, wherein the germicidal controller includes a germicidal spray source, the memory further comprising an instruction executed by the circuitry to cause the container to control the germicidal controller to spray germicide from the germicidal spray source after the entry panel of the container is closed.

5. The container of claim 1, wherein the open/close controller includes a lock, the memory further comprising an instruction executed by the circuitry to cause the container to control the open/close controller to activate the lock when the entry panel of the container is closed.

6. The container of claim 1, the memory further comprising an instruction executed by the circuitry to:
compare the first unique identifier to a value created when an order for the item was made; and
verify the first unique identifier when the first unique identifier is equal to the value.

7. The container of claim 1, the memory further comprising an instruction executed by the circuitry to:
compare the second unique identifier to a value created when an order for the item was made; and
verify the second unique identifier when the second unique identifier is equal to the value.

8. The container of claim 1, wherein the first unique identifier is one of a QR code or a biometric scan.

9. The container of claim 1, wherein the second unique identifier is one of a QR code or a biometric scan.

10. The container of claim 1, the memory further comprising an instruction executed by the circuitry to activate an alarm when the first unique identifier included in the delivery signal is not verified.

11. A process for operating a container for secure storage of an item, the container comprising a plurality of walls configured to define an interior space of the container, an entry panel positioned in at least a portion of one of the plurality of walls and operable to open and reveal the interior space of the container, circuitry, and a memory coupled to the circuitry comprising a plurality of instructions that, when executed by the circuitry, cause the container to execute the process of:
receiving a delivery signal for an item to be inserted into the container, the delivery signal including at least a first unique identifier;
opening the entry panel if the first unique identifier included in the delivery signal is verified;
closing the entry panel after the item has been placed in the interior space of the container;
providing germicide to the interior space of the first compartment after closing the entry panel of the container;
receiving a pickup signal for an item to be removed from the container, the pickup signal including at least a second unique identifier;
opening the entry panel if the second unique identifier included in the pickup signal is verified; and
closing the entry panel after the item has been removed from the interior space of the container.

12. The process of claim 11, further comprising:
determining a type of the item placed in the interior space of the container; and
setting a temperature for the interior space of the container according to the determined type of the item.

13. The process of claim 11, wherein providing a germicide further includes activating one or more UV light sources after the entry panel of the container is closed.

14. The process of claim 11, wherein providing a germicide further includes spraying germicide from a germicidal spray source after the entry panel of the container is closed.

15. The process of claim 11, further comprising activating a lock when the entry panel of the container is closed.

16. The process of claim 11, further comprising:
comparing the first unique identifier to a value created when an order for the item was made; and
verifying the first unique identifier when the first unique identifier is equal to the value.

17. The process of claim 11, further comprising:
comparing the second unique identifier to a value created when an order for the item was made; and
verify the second unique identifier when the second unique identifier is equal to the value.

18. The process of claim 11, wherein the first unique identifier is one of a QR code or a biometric scan.

19. The process of claim 11, wherein the second unique identifier is one of a QR code or a biometric scan.

20. The process of claim 11, further comprising activating an alarm when the first unique identifier included in the delivery signal is not verified.
